(19) United States
(12) Patent Application Publication
(10) Pub. No.: US 2026/0144578 A1
(43) Pub. Date: May 28, 2026

(54) INTRAMEDULLARY FIBULAR NAIL WITH ADDITIONAL FIXATION

(71) Applicant: ARTHREX, INC., Naples, FL (US)

(72) Inventors: Victoria N. Triaga, Naples, FL (US); Alexander M. DelMonaco, Chelmsford, MA (US); Manish Thapa, North Grafton, MA (US); Christopher W. Hodgkins, Belleair, FL (US); Justin J. Fleming, Richboro, PA (US)

(21) Appl. No.: 19/402,034

(22) Filed: Nov. 26, 2025

Related U.S. Application Data
(60) Provisional application No. 63/725,935, filed on Nov. 27, 2024.
(60) Provisional application No. 63/810,651, filed on May 22, 2025.

(51) Int. Cl. A61B 17/72; A61B 17/84; A61B 17/92

(52) U.S. Cl. CPC A61B 17/7241; A61B 17/846; A61B 17/921

(57) ABSTRACT

Disclosed herein are components, systems, and methods to secure an intramedullary implant to multiple fragments of a fractured bone and to secure an intramedullary implant to a fragment of a fractured bone using multiple modalities. The intramedullary implant includes a proximal body portion with transverse fixation holes positioned to receive bone fixators that secure a first bone fragment of the fractured bone to the intramedullary implant, a distal body portion with a deployable bone fixator that abuts an inner surface of a second bone fragment of the fractured bone, and a tapered intermediate body portion with a transverse fixation hole positioned to receive a bone fixator that secures the second bone fragment of the fractured bone to the intramedullary implant.

### CROSS-REFERENCE TO RELATED APPLICATION

This application claims the benefit of Provisional Application No. 63/725,935, filed Nov. 27, 2024 and Provisional Application No. 63/810,651, filed May 22, 2025, which are hereby incorporated in their entireties herein.

### BACKGROUND

Bone fractures are a common medical condition both in the young and old segments of the population. One current treatment of bone fractures includes surgically resetting the fractured bone. After the surgical procedure, the fractured area of the body (e.g., a limb in which the fractured bone is located) is often placed in an external cast for an extended period of time to ensure that the fractured bone heals properly. This can take several months for the bone to heal and for the patient to remove the cast before resuming normal activities.

In some instances, an intramedullary (IM) nail is used to align and stabilize the fracture. In that instance, a metal rod is placed inside a canal of a bone and fixed in place, typically at both ends. Placement of conventional IM nails typically require access collinear with a center line of the IM canal. For example, a fibula fracture may be treated by insertion of an IM nail. During insertion the IM nail enters a distal fragment of the fibula, is advanced across a fracture site, and enters a proximal fragment of the fibula. As used herein in reference to portions of anatomy (e.g., a bone such as a fibula), “proximal” refers to an object (e.g., a bone fragment) that is closer to the trunk or center mass of the body and “distal” refers to an object (e.g., a bone fragment that is farther from the trunk or center mass of the body.

Movement of the IM nail across the fracture site may result in movement of the distal fragment toward the proximal fragment, thereby shortening the fibula compared to its pre-fracture length. Maintaining the pre-fracture length of the fibula after the IM nail is inserted and prior to insertion of bone fixators to secure the IM nail to the distal and proximal fragments of the fibula may improve outcomes for treatment of fibular fractures.

With the distal and proximal fragments in a desired position and orientation relative to one another, one or more fixation elements are typically used to secure the distal and proximal fragments throughout the healing process. Maintaining the desired position and orientation of the distal and proximal fragments during the healing process may improve outcomes for treatment of fibular fractures.

### BRIEF SUMMARY

Some embodiments described herein provide an intramedullary implant with additional fixation elements that provide additional options for securing the intramedullary implant to multiple fragments of a fractured bone. Conventional IM fibular nails typically include a deployable fixator (e.g., one or more fingers or talons) that are inserted in a closed/insertion configuration having a narrow profile. Once positioned within an IM cavity (e.g., after crossing a fracture site), the deployable fixator may be “deployed” (e.g., transitioned from the insertion configuration to a deployed configuration). In the deployed configuration the deployable fixator may have a wider profile. For example, in the insertion configuration the deployable fixator may be movable within the IM cavity of the fractured bone and in the deployed configuration the deployable fixator may abut the inner wall of the IM cavity of the fractured bone, thereby resisting movement of the IM nail relative to the fractured bone.

Conventional IM nails that rely solely on a deployable fixator positioned within the IM cavity for fixation relative to one or more of the fragments of a fractured bone may experience some amount of movement or slippage of the conventional IM nail relative to the bone fragment. This movement during fixation of the IM nail, or during the healing process may negatively impact the final result of the treatment of the fractured bone.

Advantageously, some embodiments of an intramedullary implant disclosed herein comprise features that improve/enhance fixation. For example, an intramedullary implant may comprise a proximal body portion, a distal body portion, and an intermediate body portion between the proximal and distal body portions. The proximal body portion extends along a first central axis and defines a plurality of transverse fixation holes that each extend through the proximal body portion and intersect the first central axis. The distal body portion extends along a second central axis that is angularly offset from the first central axis, and the distal body portion comprises a deployable fixation element that transitions from an insertion configuration to a deployed configuration. In the deployed configuration, a portion of the deployable fixation element is farther from the second central axis than the portion is from the second central axis in the insertion configuration. The intermediate body portion defines a transverse fixation hole that extends through the proximal body portion and intersects the first central axis.

The proximal body portion defines a first maximum cross-sectional dimension measured in a first plane normal to the first central axis, and the distal body portion defines a second maximum cross-sectional dimension measured in a second plane normal to the second central axis. The intermediate body portion defines a third maximum cross-sectional dimension measured in a third plane normal to the first central axis. The second maximum cross-sectional dimension is less than the first maximum cross-sectional dimension, and the third maximum cross-sectional dimension tapers as the intermediate body portion approaches the distal body portion.

Some embodiments of an intramedullary implant described herein comprise a proximal body portion that extends along a first central axis and a distal body portion that extends along a second central axis that is angularly offset from the first central axis. The proximal body portion defines a first transverse fixation hole that extends through the proximal body portion and intersects the first central axis, a second transverse fixation hole that extends through the proximal body portion and intersects the first central axis and is angularly offset from the first transverse fixation hole, and a set of three transverse fixation holes that each extend through the proximal body portion parallel to one another and intersect the first central axis. The set of three transverse fixation holes are each angularly offset from both the first transverse fixation hole and the second transverse fixation hole.

The distal body portion comprises a deployable fixation element that transitions from an insertion configuration to a deployed configuration. In the deployed configuration, a portion of the deployable fixation element is farther from the second central axis than the portion is from the second central axis when in the insertion configuration. Each of the set of three transverse fixation holes are positioned distally of both the first transverse fixation hole and the second transverse fixation hole.

Some methods of fixating a first bone fragment relative to a second bone fragment described herein comprise advancing a distal body portion of an intramedullary implant through an opening formed in the first bone fragment and advancing the distal body portion through a medullary cavity of the first bone fragment. The method further comprises moving the distal body portion across a fracture site between the first bone fragment and the second bone fragment and advancing the distal body portion into and through a portion of a medullary cavity of the second bone fragment. According to the method, a proximal body portion of the intramedullary implant is advanced through the opening formed in the first bone fragment and advancing into the medullary cavity of the first bone fragment.

The method may comprise deploying a fixation element of the distal body portion within the medullary cavity of the second bone fragment until the fixation element contacts an inner wall of the second bone fragment that defines the medullary cavity of the second bone fragment. According to the method, a first bone fixation element is inserted through an exterior surface of the second bone fragment and through a first transverse fixation hole defined by the intramedullary implant and a second bone fixation element is inserted through an exterior surface of the first bone fragment and through a second transverse fixation hole defined by the intramedullary implant.

Some embodiments described herein provide a multi-lumen cannula that establishes respective pathways for each of a pair of temporary bone fixators (e.g., K-wires, guide wires, pins, etc.) into a fragment of a fractured bone. The multi-lumen cannula may be receivable in a targeting guide secured to an implant such that the pair of temporary bone fixators bracket (e.g., pass on either side of) the implant. The pair of temporary bone fixators may be inserted into the fragment to secure the targeting guide and the implant relative to the fragment. A force may then be applied to the targeting guide to move the fragment (e.g., relative to another fragment of the fractured bone) so as to achieve a desired length and/or desired orientation. The pair of temporary bone fixators may then be further advanced (e.g., into a second bone) to secure the fragment. The implant may then be secured to both fragments of the fractured bone via one or more permanent bone fixators (e.g., via bone screws, deployable talons, sutures, flexible wires, etc.).

The multi-lumen cannula may further establish respective pathways for one or more of the permanent bone fixators through a fixation hole of the implant and into the fragment of the fractured bone. When the implant is secured to both fragments of the fractured bone, the pair of temporary bone fixators may be removed from the fragment of the fractured bone. The multi-lumen cannula may be removed from the targeting guide, and the targeting guide may be decoupled from the implant. Upon completion of such a procedure using the multi-lumen cannula, only the implant and the permanent bone fixators remain secured to the fractured bone.

Insertion of an IM nail across a fracture site and into the two fragments of a fractured bone may push the two fragments together. This relative movement of the fragments may result in the fractured bone having a reduced length compared to its pre-fracture condition. Securing the fragments (e.g., via an IM nail) with the fragments pushed together may result in complications or undesired outcomes. Thus, embodiments of the multi-lumen cannula described herein may be used as a fracture reduction sleeve to provisionally secure the IM nail relative to one of the fragments of the fractured bone. A force may then be applied to the secured fragment (e.g., via a targeting guide with a recess through which the multi-lumen cannula is inserted) to move the secured fragment relative to an unsecured fragment of the fractured bone.

The multi-lumen cannula may include a first lumen, a second lumen, and a third lumen that each extend through a body of the multi-lumen cannula along respective axes. The first axis of the first lumen may be spaced apart from the second axis of the second lumen by a width sufficient to accommodate a width of the IM nail therebetween. The third axis of the third lumen may be aligned with a bone fixation hole of the IM nail. The first and second lumen being spaced apart so as to bracket the IM nail advantageously secure the multi-lumen cannula relative to the fragment of the fractured bone such that application of the force results in little to no rotational movement (e.g., movement other than axial movement parallel to the IM canal) of the of the fragment.

Some embodiments described herein provide provisional fixation of the IM nail (e.g., via the temporary fixators) to one bone fragment (e.g., a distal fragment) without blocking a path of an actuator that advances along a length of the IM nail to engage and deploy the internal fixators. The internal fixators may be talons that contact inner walls of another fragment (e.g., a proximal fragment) of the fractured bone to secure the IM nail to the previously unsecured fragment of the fracture bone.

Some embodiments of the multi-lumen cannula described herein may be reversibly receivable within a recess of a targeting guide. The multi-lumen cannula being reversibly receivable may enable consistent placement of the temporary fixators while providing multiple insertion options for the permanent fixator. For example, the multi-lumen cannula may be receivable in the recess in a first orientation to achieve a static fixation of the IM nail to the fractured bone, and may further be receivable in the recess in a second orientation to apply compression to the fixation of the IM nail to the fractured bone.

The position of the first lumen and the second lumen (that receive the temporary fixators) may be consistent relative to the targeting guide in both the first orientation and the second orientation. The position of the third lumen (that receives the permanent fixator) changes relative to the targeting guide from the first orientation to the second orientation. For example, in the first orientation the third lumen may be closer to the fracture site, and in the second orientation the third lumen may be farther from the fracture site, or vice versa.

Some embodiments of a method of using of a multi-lumen cannula described herein comprises inserting a distal end of a multi-lumen cannula through a recess of a targeting guide. The method further comprises advancing the distal end toward an implant secured to the targeting guide, advancing a first bone fixator along a first linear axis of a first lumen extending through a body of the multi-lumen cannula, and advancing a second bone fixator along a second linear axis of a second lumen extending through the body of the multi-lumen cannula. The method further includes bracketing the implant between the first bone fixator and the second bone fixator with respect to a direction perpendicular to the first linear axis. A third bone fixator is advanced along a third linear axis of a third lumen extending through the body of the multi-lumen cannula according to the method, and the third bone fixator is advanced into a through hole of the implant.

Some embodiments of the method may include positioning the implant in an intramedullary canal of a bone such that a first portion of the implant is in a proximal fragment of the bone and a second portion of the implant is in a distal fragment of the bone. The proximal fragment is separated from the distal fragment by a fracture. The method may include advancing the first bone fixator along the first linear axis of the first lumen and into the distal fragment, and advancing the second bone fixator along the second linear axis of the second lumen and into the distal fragment. A force may be applied to the multi-lumen cannula, the targeting guide, the first bone fixator, the second bone fixator, or any combination thereof as part of the method, thereby moving the distal fragment away from the proximal fragment.

Some embodiments of a multi-lumen cannula described herein comprise a proximal end, a distal end, and a body extending therebetween such that the body is symmetrical about a first plane. The multi-lumen cannula further comprises a first lumen extending through the body along a first lumen axis parallel to and offset from the first plane and a second lumen extending through the body along a second lumen axis parallel to the first lumen axis and offset from the first plane. A third lumen of the multi-lumen cannula extends through the body along a third lumen axis parallel to the first lumen axis and positioned within the first plane. The first lumen axis and the second lumen axis are positioned within a second plane perpendicular to the first plane.

Some embodiments of a multi-lumen cannula described herein comprise a proximal end, a distal end, and a body extending therebetween. A first lumen of the multi-lumen cannula extends through the body from the proximal end to the distal end along a first lumen axis positioned within a first plane. A second lumen of the multi-lumen cannula extends through the body from the proximal end to the distal end along a second lumen axis. The second lumen axis is parallel to the first lumen axis and is positioned within the first plane offset from the first lumen axis in a first direction that is perpendicular to the first lumen axis. A third lumen of the multi-lumen cannula extends through the body from the proximal end to the distal end along a third lumen axis that is parallel to the first lumen axis. The third lumen axis is positioned within a second plane that is parallel to the first plane and offset from the first plane in a second direction that is perpendicular to the first plane. The first lumen and the second lumen each are positioned between the second plane and a third plane that is parallel to the second plane and tangent to the third lumen.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not necessarily drawn to scale, and some of these elements may be arbitrarily enlarged and positioned to improve drawing legibility. Further, the particular shapes of the elements as drawn, are not necessarily intended to convey any information regarding the actual shape of the particular elements, and may have been solely selected for ease of recognition in the drawings. The headings and Abstract of the Disclosure provided herein are for convenience only and do not interpret the scope or meaning of the embodiments.

FIG. 1 is an anterior view of a fractured fibula.

FIG. 2 is an anterior view of the fractured fibula illustrated in FIG. 1 with the fractured fibula being prepared to receive an intramedullary implant.

FIG. 3 is an anterior view of the fractured fibula illustrated in FIG. 2 with an intramedullary implant being inserted into an intramedullary canal of the fractured fibula and a targeting guide attached to the intramedullary implant.

FIG. 4 is a top, first side isometric view of an intramedullary implant.

FIG. 5 is a bottom, second side isometric view of the intramedullary implant illustrated in FIG. 4.

FIG. 6 is a cross-sectional view of the intramedullary implant illustrated in FIG. 4 in a first orientation with a deployable fixator in an insertion configuration.

FIG. 7 is a cross-sectional view of the intramedullary implant illustrated in FIG. 6 in the first orientation with the deployable fixator in a deployed configuration.

FIG. 8 is a cross-sectional view of the intramedullary implant illustrated in FIG. 4 in a second orientation with the deployable fixator in an insertion configuration.

FIG. 9 is a cross-sectional view of the intramedullary implant illustrated in FIG. 8 in the second orientation with the deployable fixator in a deployed configuration.

FIG. 10 is a bottom plan view of the intramedullary implant illustrated in FIG. 4.

FIG. 11 is a top, first side isometric view of another intramedullary implant.

FIG. 12 is a lateral view of the fractured fibula, intramedullary implant, and targeting guide illustrated in FIG. 3 and a multi-lumen cannula in a first orientation.

FIG. 13 is a lateral view of the fractured fibula, intramedullary implant, targeting guide, and multi-lumen cannula illustrated in FIG. 12, and temporary bone fixators inserted into a first fragment of the fractured fibula.

FIG. 14 is a lateral view of the fractured fibula, intramedullary implant, targeting guide, multi-lumen cannula, and temporary bone fixators illustrated in FIG. 13, with the multi-lumen cannula in a second orientation.

FIG. 15 is a posterior-lateral view of the fractured fibula, intramedullary implant, targeting guide, and multi-lumen cannula illustrated in FIG. 14, with temporary bone fixators inserted into a first fragment of the fractured fibula.

FIG. 16 is a medial view of the fractured fibula, intramedullary implant, targeting guide, and temporary bone fixators illustrated in FIG. 15.

FIG. 17 is an anterior view of the fractured fibula, intramedullary implant, targeting guide, multi-lumen cannula, and temporary bone fixators illustrated in FIG. 15, with a force being applied to the first fragment of the fractured bone.

FIG. 18 is an anterior view of the fractured fibula, intramedullary implant, targeting guide, multi-lumen cannula, and temporary bone fixators illustrated in FIG. 17, with an actuator deploying internal fixators of the intramedullary implant.

FIG. 19 is an anterior view of the fractured fibula, intramedullary implant, targeting guide, multi-lumen cannula, and temporary bone fixators illustrated in FIG. 18, with a permanent bone fixator securing the intramedullary implant to the first fragment of the fractured bone and a second permanent bone fixator securing the intramedullary implant to the second fragment of the fractured bone.

FIG. 20 is an anterior view of the fractured fibula, intramedullary implant, targeting guide, illustrated in FIG. 19, with permanent bone fixators securing the intramedullary implant to the first fragment of the fractured bone.

FIG. 21 is an anterior view of the fractured fibula and intramedullary implant illustrated in FIG. 20, with permanent bone fixators securing the intramedullary implant to the first and second fragments of the fractured bone and an end cap being secured to the intramedullary implant.

FIG. 22 is an anterior view of the intramedullary implant illustrated in FIG. 21 secured within the fractured fibula upon completion of a surgical procedure to repair the fractured fibula.

### DETAILED DESCRIPTION

As noted above, conventional intramedullary implants (e.g., fibular nails) typically include a deployable fixator (e.g., one or more fingers or talons) that are inserted into an intramedullary canal (e.g., along a length of the intramedullary canal) in an insertion configuration having a narrow profile. Once positioned within an IM cavity (e.g., after crossing a fracture site), the deployable fixator may be “deployed” (e.g., transitioned from the insertion configuration to a deployed configuration). In the deployed configuration the deployable fixator may have a wider profile. For example, in the insertion configuration the deployable fixator may be movable within the IM cavity of the fractured bone and in the deployed configuration the deployable fixator may abut the inner wall of the IM cavity of the fractured bone, thereby resisting movement of the IM nail relative to the fractured bone.

Conventional IM nails that rely solely on a deployable fixator positioned within the IM cavity for fixation relative to one or more of the fragments of a fractured bone may experience some amount of movement or slippage of the conventional IM nail relative to the bone fragment. This movement during fixation of the IM nail, or during the healing process may negatively impact the final result of the treatment of the fractured bone.

Advantageously, some embodiments of the intramedullary implant disclosed herein comprise improved fixation. For example, the intramedullary implant may comprise transverse fixation holes that each extend through the intramedullary implant. The transverse fixation holes may be positioned such that, when inserted in an IM canal, at least one of the transverse fixation holes is positioned within a first bone fragment (e.g., on one side of a fracture of the bone) and another of the transverse fixation holes is positioned within a second bone fragment (e.g., on the other side of the fracture of the bone). This arrangement enables additional fixation (e.g., a bone fixator such as a screw) that cooperates with the deployable fixator such that the deployable fixator is not the only source of fixation between the first bone fragment and the intramedullary implant.

Some embodiments of the intramedullary implant described herein may be included as part of a kit. The kit may comprise one or more of the intramedullary implant (e.g., in various sizes, lengths, widths, etc.), a targeting guide that assists in placement of temporary and/or permanent fixators (e.g., bone screws) to secure the intramedullary implant relative to a fractured bone, a multi-lumen cannula, an actuator, drills, reamers, or any combination thereof.

Conventional targeting guides are known and utilized to secure hardware, such as intramedullary nails, to bone. Some known intramedullary implants push together fragments of a fractured bone separated by a fracture. Securing the fragments of the fractured bone when pushed together may result in a shortening of the fractured bone (e.g., compared to its pre-fractured condition and/or compared to a corresponding bone on the other side of the body, such as a fibula).

Some embodiments of the multi-lumen cannula described herein advantageously enable temporary bone fixators to be advanced into a first fragment of the fractured bone such that the temporary bone fixators are supported along a portion (e.g., at least a majority) of their length between a targeting guide that receives the multi-lumen cannula and the fractured bone. With the length of the temporary bone fixators supported, a force can be applied to the first fragment of the fractured bone (e.g., via the targeting guide and the supported temporary bone fixators) to move the first fragment relative to a second fragment of the fractured bone so as to achieve a desired length and/or a desired orientation.

In some cases, the multi-lumen cannula defines respective paths for the temporary bone fixators to pass on either side of (e.g., bracket) the intramedullary implant, without passing through the intramedullary implant. Thus, the multi-lumen cannula may be used as a fracture reduction sleeve that advantageously provisionally secures the intramedullary implant relative to the first bone fragment without blocking a path through the intramedullary implant along which an actuator travels to engage and deploy an internal fixator of the intramedullary implant. The internal fixator may be positioned within the second fragment of the fractured bone such that upon deployment of the internal fixator the internal fixator engages an inner wall of the second fragment thereby securing the intramedullary implant to the second fragment.

Conventional bone fixation systems include guides that provide provisional fixation via a temporary bone fixator that passes through or along one side of an intramedullary implant. As described above, passage through the intramedullary implant blocks an actuator from reaching a far end of the intramedullary implant to deploy internal bone fixators. Unbalanced temporary bone fixators (e.g., on one side of the intramedullary implant) may result in twisting or rotational forces being applied to the first bone fragment, which may, detrimentally, move the first bone fragment out of alignment with the second bone fragment.

Some known bone fixation systems provide a curved path for the temporary bone fixator to avoid contact with the intramedullary implant. However, the curved path may result in less stable fixation. Advantageously, the linear lumens of some embodiments of the disclosed multi-lumen cannula enable the temporary bone fixators to be advanced into the first bone fragment, and then subsequently advanced again (e.g., further into the first bone fragment and/or into a second bone (e.g., different than the fractured bone) to provisionally secure the first bone fragment to the second bone until permanent bone fixators are inserted.

Depending on the specific bone fractured, and the location of the fracture within the specific bone, one or more of the permanent fixators may have options for their placement through the intramedullary implant. For example, a static fixation may be achieved by insertion of a bone screw through a first portion of a bone fixation hole in the intramedullary implant, and a compression fixation may be achieved by insertion of the bone screw through a second portion of the bone fixation hole.

Advantageously, some embodiments of the multi-lumen cannula may be symmetrical about a plane of symmetry. The multi-lumen cannula may be insertable into a recess of the targeting guide in two different orientations. The locations of the first lumen and the second lumen (and the resulting location of the temporary bone fixator inserted therethrough) relative to the first fragment of the fractured bone may be the same in both of the two orientations. For example, the first lumen and the second lumen may swap positions when transitioning from the first orientation to the second orientation, such that the two temporary bone fixators collectively are in the same location.

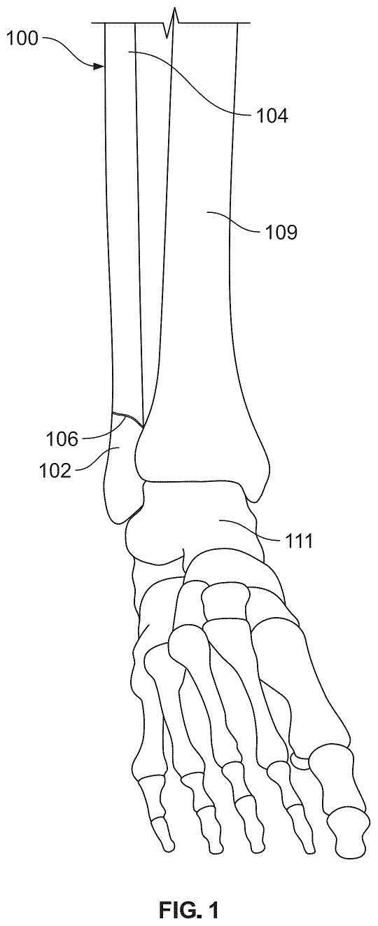

Referring now to the drawings, and specifically to FIGS. 1 to 3, a fractured bone 100 may comprise two or more fragments (e.g., a first bone fragment 102 and a second bone fragment 104) separated by one or more fractures 106. For example, the fractured bone 100 may be a fibula, the first bone fragment 102 may be a distal fragment, and the second bone fragment 104 may be a proximal fragment. As shown in FIG. 1, prior to insertion of any fixation hardware, the first bone fragment 102 and the second bone fragment 104 may be aligned (e.g., to approximate the shape of the fractured bone 100 prior to the fracture 106—referred to herein as the “pre-fracture condition”). A clamp (not shown) may be used to hold the first bone fragment 102, the second bone fragment 104, or both in place.

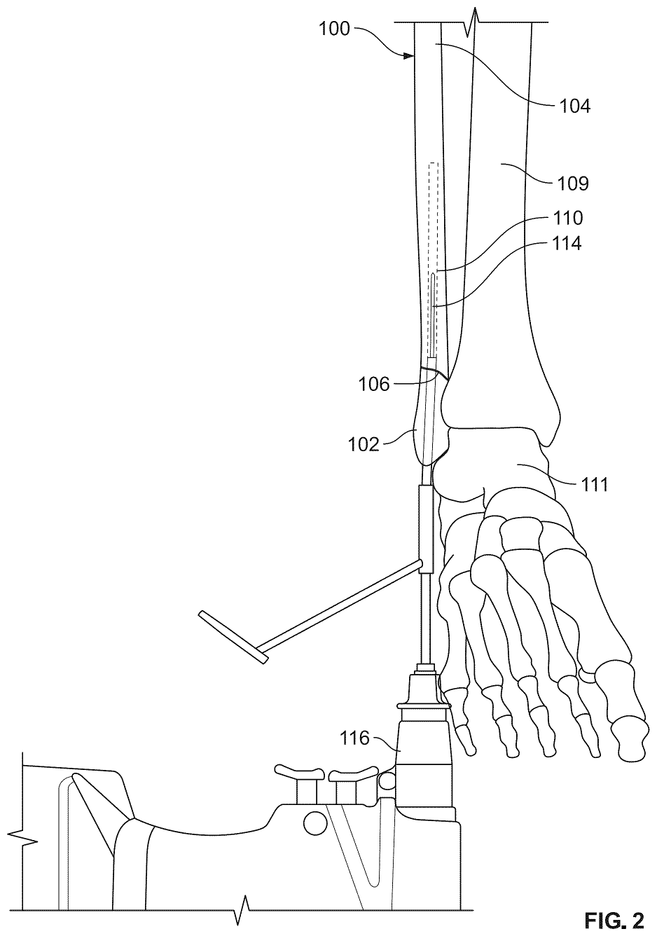

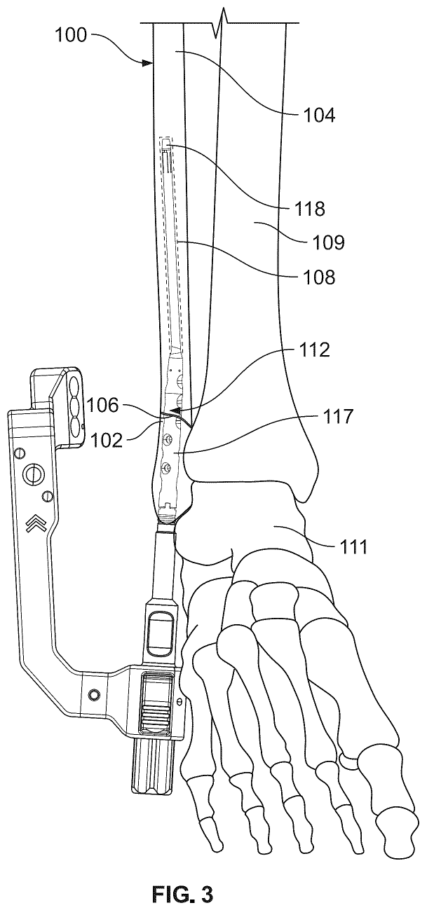

As shown in FIGS. 2 and 3, instruments may be used to form a hole 108 within the fractured bone 100. The hole 108 may include an intramedullary canal 110 of the fractured bone 100. The intramedullary canal 110 may be enlarged along at least a portion of the length of the fractured bone 100 to a size that can accommodate an intramedullary implant 112. For example, a guide wire 114 may be inserted through a surface in the first bone fragment 102 (e.g., a distal surface of the lateral malleolus) and into the intramedullary canal 110. The guide wire 114 may be advanced (e.g., proximally/anteriorly) across the fracture 106 into the intramedullary canal 110 of the second bone fragment 104. A drill or reamer 116 may be used to form the hole 108 (e.g., by enlarging the intramedullary canal 110 to a desired size). The drill or reamer 116 may be cannulated and inserted over the guide wire 114 and advanced along the guide wire 114 through the first bone fragment 102, across the fracture 106, and into the second bone fragment 104.

Once the hole 108 is formed, the intramedullary implant 112 (e.g., an intramedullary nail 117) may be positioned within the hole 108. For example, a tip 118 of the intramedullary nail 117 may be advanced (e.g., proximally/anteriorly) through a surface in the first bone fragment 102 (e.g., the distal surface of the lateral malleolus), across the fracture 106, and into the second bone fragment 104. Once positioned in the hole 108 as desired, the intramedullary nail 117 may be secured to both the first bone fragment 102 and the second bone fragment 104 of the fractured bone 100, as described in further detail below.

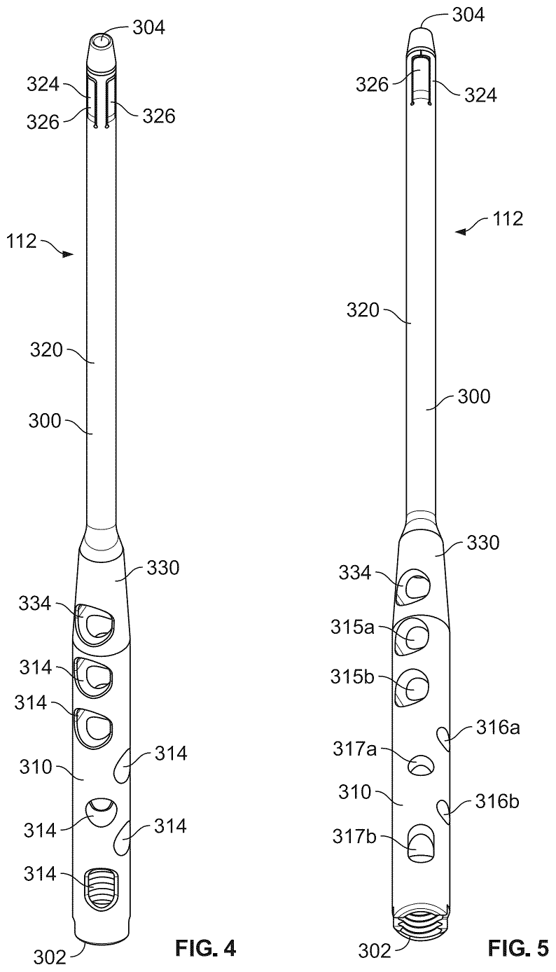

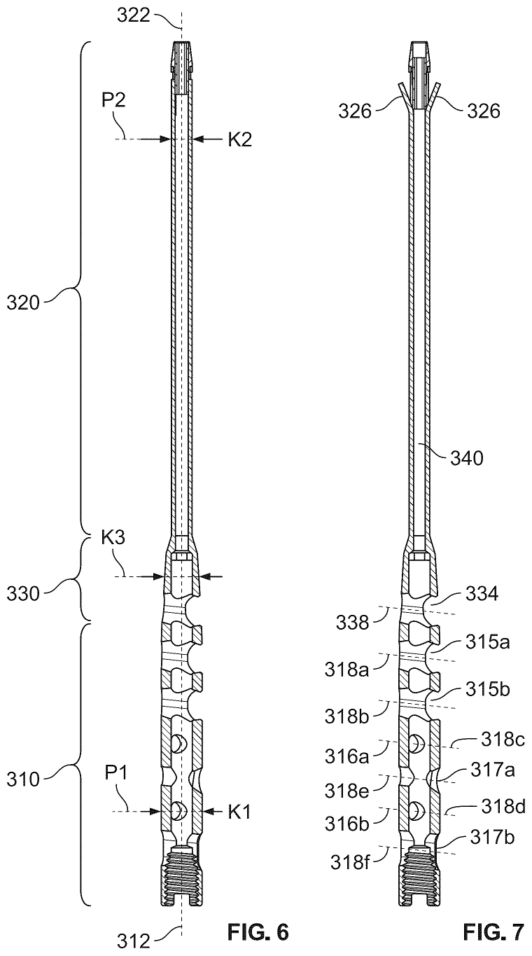

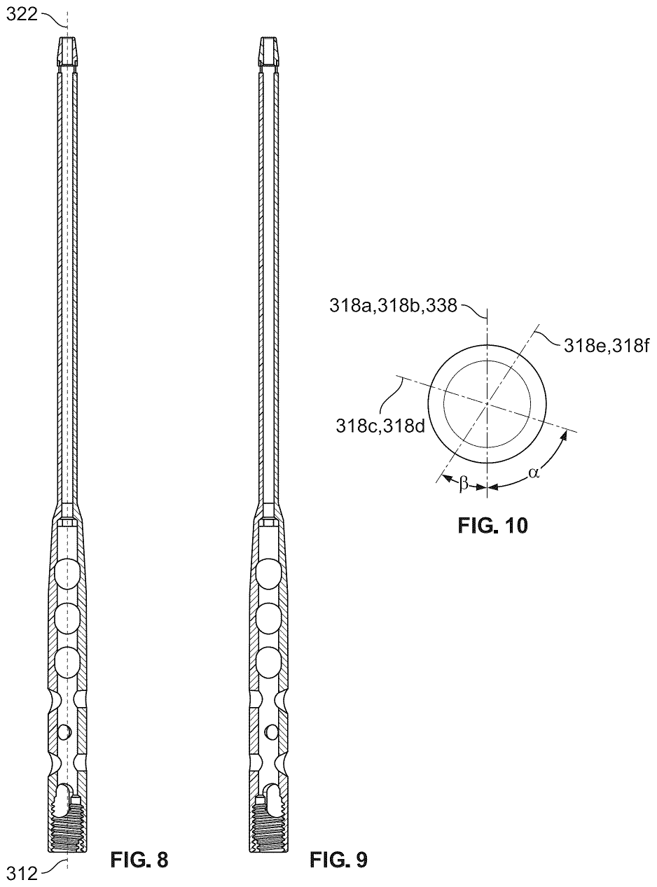

Referring to FIGS. 4 to 11, some embodiments of the intramedullary implant 112 described herein comprise an implant body 300 that extends from a proximal end 302 to a distal end 304. The implant body 300 may comprise a proximal body portion 310 that extends along a first central axis 312. The proximal body portion 310 may define a plurality of transverse fixation holes 314 that each extend through the proximal body portion 310 and intersect the first central axis 312. As shown, the proximal body portion 310 may define a first maximum cross-sectional dimension K1 measured in a first plane P1 that is normal to the first central axis 312. The proximal body portion 310 may be cylindrical or another shape with a constant cross-sectional dimension K1 along at least a portion of a length of the proximal body portion 310. According to some embodiments, one or more portions up to an entirety of the proximal body portion 310 may be tapered (e.g., having a maximum cross-sectional dimension that decreases distally).

The plurality of transverse fixation holes 314 may comprise multiple subsets of transverse fixation holes (e.g., a first subset of transverse fixation holes 315, a second subset of transverse fixation holes 316, and a third subset of transverse fixation holes 317). Each of the multiple subsets may comprise a single transverse fixation hole 314, or may comprise multiple (e.g., two or more, for example between two and five) transverse fixation holes 314. As shown, each of the plurality of transverse fixation holes 314 within a given subset may be parallel to one another and angularly offset from those of the plurality of transverse fixation holes 314 within a different subset.

For example, the first subset of transverse fixation holes 315 may comprise a distal-most one of the plurality of transverse fixation holes 314. According to some embodiments, the first subset of transverse fixation holes 315 may comprise a first transverse fixation hole 315a (e.g., the distal-most one of the plurality of transverse fixation holes 314) and a second transverse fixation hole 315b, positioned proximally of the first transverse fixation hole 315a.

The second subset of transverse fixation holes 316 may comprise at least one of the plurality of transverse fixation holes 314 positioned proximally of the first transverse fixation hole 315a. As shown, the second subset of transverse fixation holes 316 may comprise a third transverse fixation hole 316a and a fourth transverse fixation hole 316b, positioned proximally of the third transverse fixation hole 316a. According to some embodiments, the third transverse fixation hole 316a and the fourth transverse fixation hole 316b may both be positioned proximally of both the first transverse fixation hole 315a and the second transverse fixation hole 315b.

The third subset of transverse fixation holes 317 may comprise at least one of the plurality of transverse fixation holes 314 positioned proximally of the third transverse fixation hole 316a. As shown, the third subset of transverse fixation holes 317 may comprise a fifth transverse fixation hole 317a and a sixth transverse fixation hole 317b, positioned proximally of the fifth transverse fixation hole 317a. According to some embodiments, the second subset of transverse fixation holes 316 and the third subset of transverse fixation holes 317 may be arranged alternatingly. For example, the plurality of transverse fixation holes 314 may be arranged distal to proximal as follows: the first transverse fixation hole 315a, the second transverse fixation hole 315b, the third transverse fixation hole 316a, the fifth transverse fixation hole 317a, the fourth transverse fixation hole 316b, and the sixth transverse fixation hole 317b.

Some embodiments of the intramedullary implant 112 may comprise a distal body portion 320 that extends along a second central axis 322 that is either aligned with or angularly offset from the first central axis 312. As shown, the first central axis 312 and the second central axis 322 may be angularly offset within a range of 0 degrees to 25 degrees, e.g., from 2 degrees to 20 degrees, from 3 degrees to 15 degrees, from 4 degrees to 10 degrees, or from 5 degrees to 8 degrees. In terms of lower limits, the first central axis 312 and the second central axis 322 may be angularly offset by greater than 2 degrees, e.g., greater than 3 degrees, greater than 4 degrees, greater than 5 degrees, greater than 6 degrees, greater than 8 degrees, greater than 10 degrees, greater than 15 degrees, or greater than 20 degrees. In terms of upper limits, the first central axis 312 and the second central axis 322 may be angularly offset by less than 25 degrees, e.g., less than 20 degrees, less than 15 degrees, less than 10 degrees, less than 8 degrees, less than 6 degrees, less than 5 degrees, less than 4 degrees, or less than 3 degrees. For example, the first central axis 312 and the second central axis 322 may be angularly offset by about 6 degrees (e.g., plus or minus 2 degrees). The distal body portion 320 may comprise a deployable fixation element 324 that transitions from an insertion configuration to a deployed configuration. For example, the deployable fixation element 324 may include one or more movable talons 326 or fingers that transition from an insertion configuration with a narrower profile to a deployed configuration with a wider profile.

In the insertion configuration, the one or more movable talons 326 may be positioned closer to the second central axis 322 resulting in the narrower profile. The narrow profile may enable the deployable fixation element 324 and the distal body portion 320 to be advanced through a hole formed in the intramedullary canal of a fractured bone. When the distal body portion 320 is positioned within a portion of a hole (e.g., the hole 108 formed in the second bone fragment 104 of the fractured bone 100), the deployable fixation element 324 may transition to the deployed configuration.

In the deployed configuration, at least a portion of the one or more movable talons 326 moves away from the second central axis 322 thereby defining the wider profile. In the deployed configuration one or more of the movable talons 326 may directly abut an inner surface of the second bone fragment 104 (e.g., that forms a perimeter of the hole 108). Abutment of the deployable fixation element 324 with the second bone fragment 104 may restrict movement of the distal body portion 320 relative to the second bone fragment 104.

In the insertion configuration, the distal body portion 320 (e.g., the deployable fixation element 324) may define a second maximum cross-sectional dimension K2 measured in a second plane P2. The second plane P2 may be normal to the second central axis 322 and/or may be parallel to the first plane P1. The distal body portion 320 may be devoid of any transverse fixation holes, as shown in the illustrated embodiment. Alternatively, one or more transverse fixation holes may be formed in the distal body portion 320.

As used herein when referring to portions of the intramedullary implant 112, “proximal” refers to a portion (e.g., a proximal body portion) that is closer to the surgeon implanting the intramedullary implant 112 and/or closer to an incision through which the intramedullary implant 112 was inserted when fully inserted and “distal” refers to a portion (e.g., a distal body portion) that is farther from the surgeon implanting the intramedullary implant 112 and/or closer to an incision through which the intramedullary implant 112 was inserted when fully inserted. The distal/proximal of the anatomy may not match the distal/proximal of the intramedullary implant 112 (e.g., the proximal body portion 310 may be secured within a distal bone fragment and the distal body portion 320 may be secured within a proximal bone fragment).

Some embodiments of the intramedullary implant 112 may comprise an intermediate body portion 330 between the proximal body portion 310 and the distal body portion 320. The intermediate body portion 330 may extend along the first central axis 312, as shown. The intermediate body portion 330 may define a transverse fixation hole 334 that extends through the intermediate body portion 330 and intersects the first central axis 312. The intermediate body portion 330 may define a third maximum cross-sectional dimension K3 measured in a third plane P3 that is normal to the first central axis 312. The third maximum cross-sectional dimension K3 may change along a length of the intermediate body portion 330 (e.g., reducing as the intermediate body portion 330 approaches the distal body portion 320).

The transverse fixation hole 334 of the intermediate body portion 330 may be parallel with at least one of the plurality of transverse fixation holes 314 of the proximal body portion 310. For example, the transverse fixation hole 334 may be parallel to the first transverse fixation hole 315a. As shown, the first transverse fixation hole 315a may be adjacent to (and proximal of) the transverse fixation hole 334 of the intermediate body portion 330, and further be adjacent to (and distal of) the second transverse fixation hole 315b of the proximal body portion 320. Accordingly, some embodiments of the intramedullary implant 112 may comprise a set of three transverse fixation holes parallel to each other and in series along the length L of the implant body 300. The set of three transverse fixation holes may comprise the transverse fixation hole 334, the first transverse fixation hole 315a, and the second transverse fixation hole 315b. The set of three transverse fixation holes may be positioned such that first and second fragments of a fractured bone may be secured to the intramedullary implant 112 via insertion of screws that are parallel to each other on either side of a fracture location (e.g., one screw inserted into a first bone fragment and another screw inserted into a second bone fragment).

The transverse fixation hole 334 may be positioned along a length L of the intramedullary implant 112 at a location that is closer to the proximal end 302 of the implant body 300 than the location is from the distal end 304 of the implant body 300. According to some embodiments, the location of the transverse fixation hole 334 may be at least 1.5 times (e.g., at least 2 times) farther from the distal end 304 than the location is from the proximal end 302. According to some embodiments, the location of the transverse fixation hole 334 may be between about 1.5 times and about 2 times farther from the distal end 304 than the location is from the proximal end 302.

As shown in FIG. 10, each of the plurality of transverse fixation holes 314 and the transverse fixation hole 334 may extend through the implant body 300 along respective hole axes. For example, the first transverse fixation hole 315a may extend along a first hole axis 318a and the second transverse fixation hole 315b may extend along a second hole axis 318b. The first hole axis 318a and the second hole axis 318b may be parallel to one another (e.g., such that when viewed from the proximal end 302 they appear colinear). Similarly, the third transverse fixation hole 316a may extend along a third hole axis 318c and the fourth transverse fixation hole 316b may extend along a fourth hole axis 318d. The third hole axis 318c and the fourth hole axis 318d may be parallel to one another (e.g., such that when viewed from the proximal end 302 they appear colinear). Similarly, the fifth transverse fixation hole 317a may extend along a fifth hole axis 318e and the sixth transverse fixation hole 317b may extend along a sixth hole axis 318f. The fifth hole axis 318e and the sixth hole axis 318f may be parallel to one another (e.g., such that when viewed from the proximal end 302 they appear colinear).

The transverse fixation hole 334 of the intermediate body portion 330 may extend along a hole axis 338. As shown the hole axis 338 may be parallel to the first hole axis 318a and the second hole axis 318b (e.g., such that when viewed from the proximal end 302 they appear colinear). The hole axis 338 may be angularly offset from the third hole axis 318c and the fourth hole axis 318d by an angle α. According to some embodiments, the angle α may range from 0 degrees to 90 degrees, e.g., from 5 degrees to 70 degrees, from 10 degrees to 50 degrees, from 15 degrees to 40 degrees, or from 20 degrees to 30 degrees. In terms of lower limits, the angle α may be greater than 5 degrees, e.g., greater than 10 degrees, greater than 15 degrees, greater than 20 degrees, greater than 30 degrees, greater than 40 degrees, greater than 50 degrees, or greater than 70 degrees. In terms of upper limits, the angle α may be less than 90 degrees, e.g., less than 70 degrees, less than 50 degrees, less than 40 degrees, less than 30 degrees, less than 20 degrees, less than 15 degrees, less than 10 degrees, or less than 5 degrees. For example, the angle α may be about 25 degrees (e.g., plus or minus 5 degrees).

The hole axis 338 may be angularly offset from the fifth hole axis 318e and the sixth hole axis 318f by an angle β. According to some embodiments, the angle β may range from 0 degrees to 90 degrees, e.g., from 10 degrees to 85 degrees, from 20 degrees to 80 degrees, from 30 degrees to 75 degrees, or from 50 degrees to 70 degrees. In terms of lower limits, the angle β may be greater than 10 degrees, e.g., greater than 20 degrees, greater than 30 degrees, greater than 40 degrees, greater than 50 degrees, greater than 70 degrees, greater than 75 degrees, greater than 80 degrees, or greater than 85 degrees. In terms of upper limits, the angle β may be less than 90 degrees, e.g., less than 85 degrees, less than 80 degrees, less than 75 degrees, less than 70 degrees, less than 50 degrees, less than 40 degrees, less than 30 degrees, less than 20 degrees, or less than 10 degrees. For example, the angle β may be about 60 degrees (e.g., plus or minus 10 degrees).

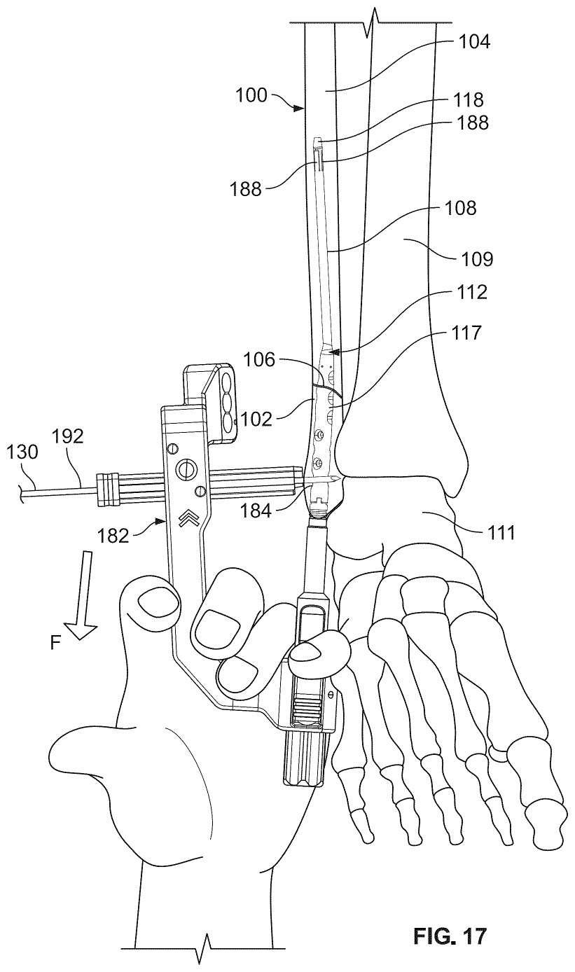

Some embodiments of the intramedullary implant 112 may comprise an axial bore 340 that extends through the proximal body portion 310, along the first central axis 312, through the intermediate body portion 330, and into the distal body portion 320 (e.g., along at least a portion of the second central axis 322). The axial bore 340 may intersect the transverse fixation hole 334 of the intermediate body portion 330 and any, up to all, of the plurality of transverse fixation holes 314 of the proximal body portion 310. The intramedullary implant 112 may comprise an actuator (e.g., the actuator 186 shown in FIG. 17) with a shaft that is sized to advance along the axial bore 340 and engage the deployable fixation element 324. The actuator may further comprise a handle accessible outside of the patient's body that can be rotated to rotate the shaft to thereby transition the deployable fixation element 324 from the insertion configuration to the deployed configuration.

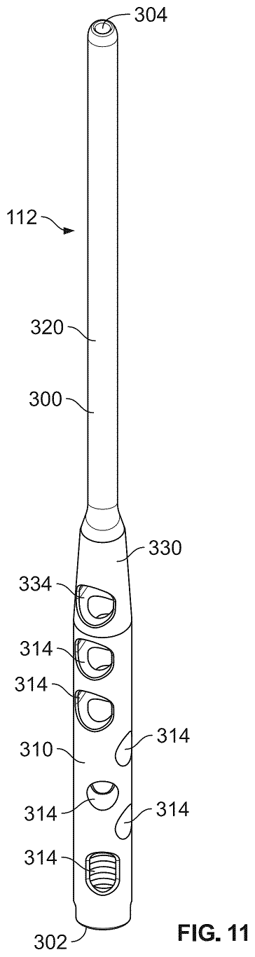

As shown in FIG. 11, some embodiments of the intramedullary implant 112 may be devoid of the deployable fixation element 324, but otherwise identical to other embodiments of the intramedullary implant 112 as described herein. Accordingly, some embodiments of the intramedullary implant 112 may be securable to the second bone fragment 104 without the use of an internal/deployable fixation element, and may instead be secured via a bone screw inserted through the transverse fixation hole 334 of the intermediate body portion 330.

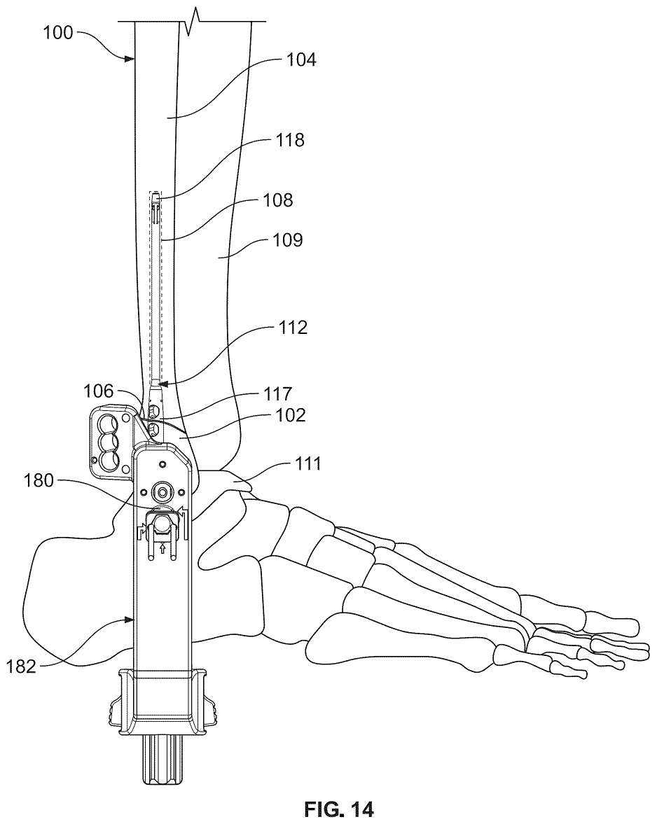

Referring to FIGS. 1 to 22, a method of fixating a first bone fragment (e.g., the first bone fragment 102) relative to a second bone fragment (e.g., the second bone fragment 104) may comprise advancing the distal body portion 320 of the intramedullary implant 112 through an opening formed in the first bone fragment 102 and through a hole (e.g., the hole 108, a medullary cavity, etc.) defined by the first bone fragment 102. The method may comprise moving the distal body portion 320 across the fracture 106 between the first bone fragment 102 and the second bone fragment 104 and advancing the distal body portion 320 into and through a portion of a medullary cavity of the second bone fragment 104.

Some embodiments of the method may further comprise advancing the proximal body portion 310 of the intramedullary implant 112 through the opening formed in the first bone fragment 102 and advancing the proximal body portion 310 into the medullary cavity of the first bone fragment 102. When the distal body portion 320 is positioned within the second bone fragment 104 the deployable fixation element 324 may be transitioned from the insertion configuration to the deployed configuration in which the movable talons 326 contact an inner wall of the second bone fragment 104. After transitioning the deployable fixation element 324, a first bone fixation element (e.g., a bone screw 350) may be inserted through an exterior surface of the second bone fragment 104 and through the transverse fixation hole 334 of the intramedullary implant 112. According to the method, a second bone fixation element (e.g., a bone fixator 194 such as a bone screw) may be inserted through an exterior surface of the first bone fragment 102 and through one of the plurality of transverse fixation holes 314 of the proximal body portion 310 of the intramedullary implant 112.

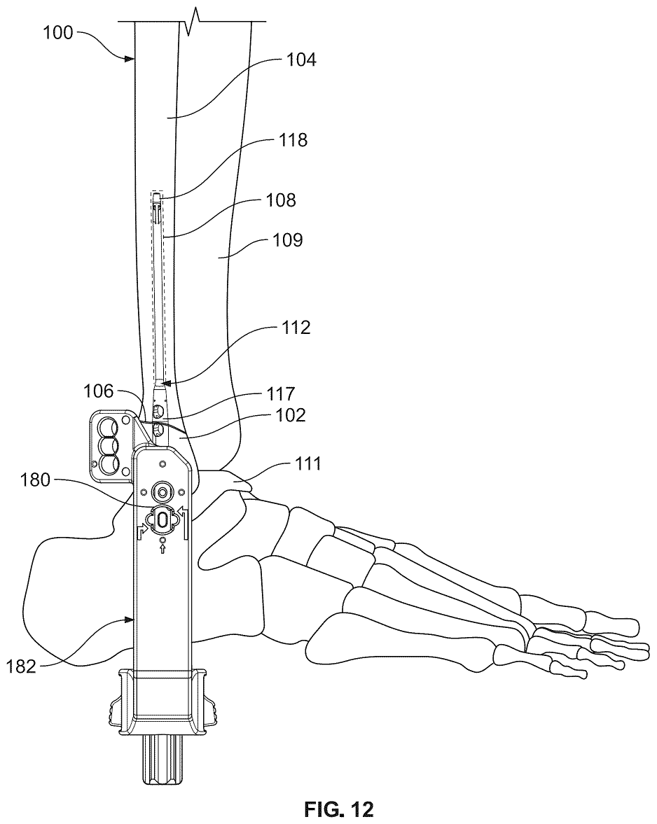

As shown in FIGS. 12 to 14, the method may comprise inserting a multi-lumen cannula 120 through a recess 180 of a targeting guide 182 that is secured to the intramedullary nail 117. The recess 180 may have a “keyed” shape that corresponds to an outer perimeter of a body 126 of the multi-lumen cannula 120. Thus, when positioned within the recess 180, the multi-lumen cannula 120 may not be rotatable relative to the targeting guide 182. The method may further include advancing the multi-lumen cannula 120 toward the intramedullary nail 117 (e.g., until a gap between the multi-lumen cannula 120 and the intramedullary nail 117 is minimized, for example below about 20 mm).

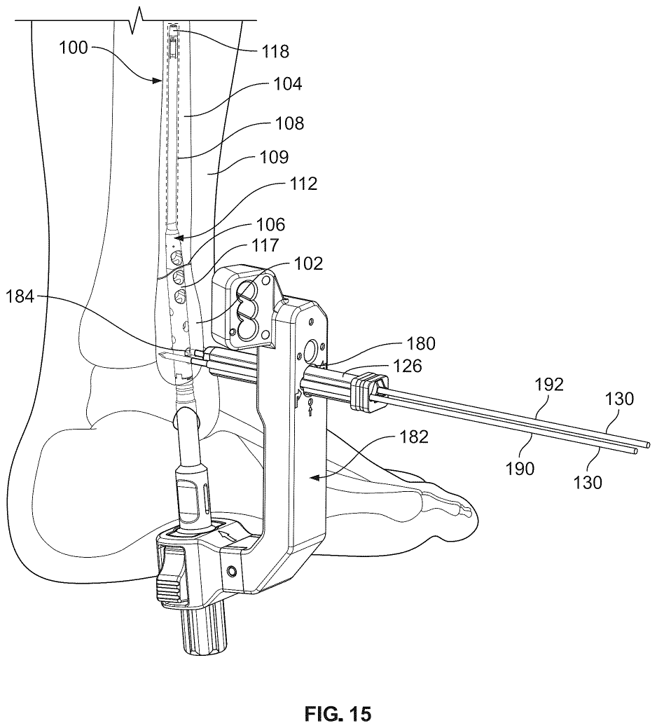

As shown in FIG. 15, the method may further include advancing a first bone fixator 190 (e.g., a K-wire) through a first lumen of the multi-lumen cannula (e.g., along a first lumen axis and advancing a second bone fixator 192 (e.g., a K-wire) through a second lumen (e.g., along a second lumen axis). When the multi-lumen cannula 120 is positioned within the recess 180 and the targeting guide 182 is secured to the intramedullary nail 117, the first bone fixator 190 may be advanced along the first lumen axis passing on one side of the intramedullary nail 117, and the second bone fixator 192 may be advanced along the second lumen axis passing on the other side of the intramedullary nail 117.

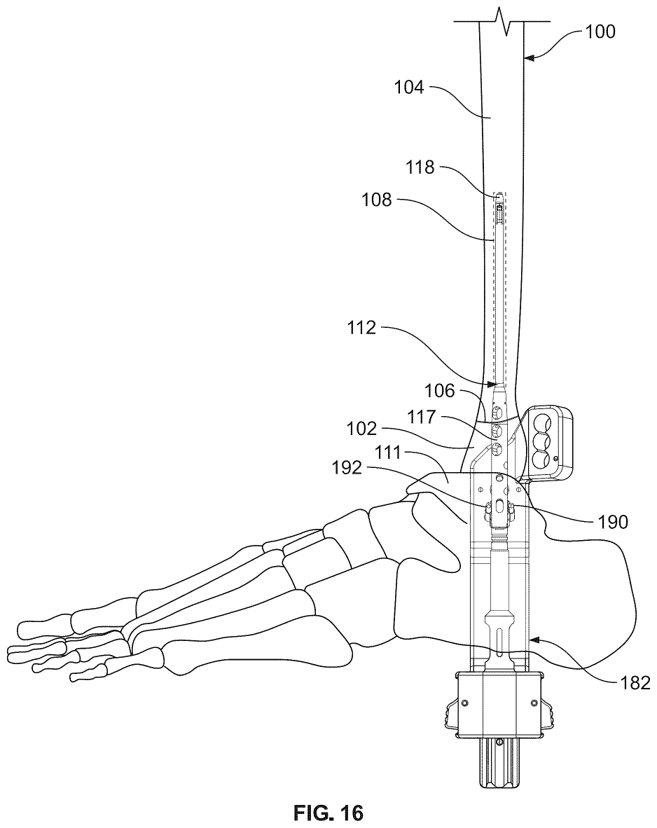

As shown in FIG. 16, the method may include bracketing the intramedullary nail 117 between the first bone fixator 190 and the second bone fixator 192. As shown in FIG. 17, the method may further include advancing a third bone fixator 194 (e.g., a bone screw) through a third lumen of the multi-lumen cannula 120 (e.g., along a third lumen axis). When the multi-lumen cannula 120 is positioned within the recess 180 and the targeting guide 182 is secured to the intramedullary nail 117, the third bone fixator 194 may be advanced along the third lumen axis until passing through a transverse fixation hole 314 of the intramedullary nail 117. According to some embodiments, the third lumen axis and the intramedullary nail 117 may be substantially perpendicular to one another (e.g., within about 10 degrees of offset). Thus, a central axis of the transverse fixation hole 314 may be perpendicular to the third lumen axis, or offset from the third lumen axis by up to about 10 degrees.

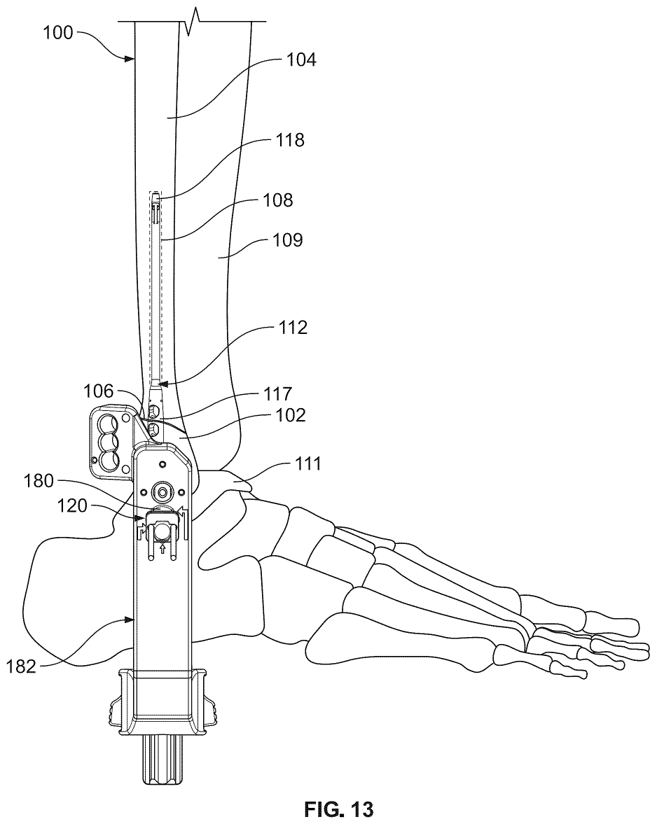

The multi-lumen cannula 120 may be in a first orientation relative to the targeting guide 182 when the multi-lumen cannula 120 is positioned within the recess 180, as shown in FIG. 13. The method may further comprise withdrawing the multi-lumen cannula 120 through the recess 180 of the targeting guide 182, rotating the multi-lumen cannula 120 (e.g., 180 degrees) to a second orientation, and then inserting the multi-lumen cannula through the recess 180 of the targeting guide 182 while the multi-lumen cannula 120 is in the second orientation. When the multi-lumen cannula 120 is positioned within the recess 180 in the second orientation, the first bone fixator 190, the second bone fixator 192, and/or the third bone fixator 194 may be inserted through the first lumen, the second lumen, and/or the third lumen, respectively, as described above in reference to the first orientation.

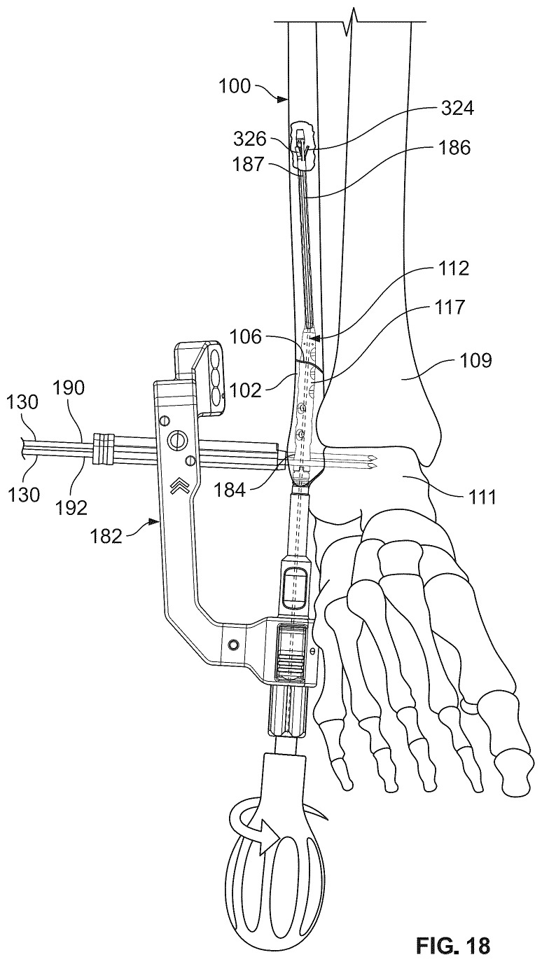

As shown in FIG. 18, the method may include advancing an actuator 186 along a length of the intramedullary nail 117, passing the actuator 186 between the first bone fixator 190 and the second bone fixator 192. According to some embodiments of the method, the actuator 186 may be advanced prior to advancing the third bone fixator 194 into the bone fastener receiving hole 184 of the intramedullary nail 117. The actuator 186 may be advanced through a channel within the intramedullary nail 117, and the channel may be blocked by the third bone fixator 194, when the third bone fixator 194 is positioned within the bone fastener receiving hole 184.

The method may include advancing an actuator 186 within the intramedullary nail 117 until the actuator 186 engages the deployable fixation element 324 of the intramedullary nail 117. The actuator 186 may be rotated within the intramedullary nail 117 while the actuator 186 is engaged with the deployable bone fixator 196 to deploy the deployable bone fixator 196 (e.g., such that the deployable bone fixator 196 engages with an inner surface of the second bone fragment 104). Once the deployable fixation element 324 is in the deployed configuration, the method may include withdrawing the actuator 186 from the intramedullary nail 117 prior to advancing the third bone fixator 194 into the transverse fixation hole 314.

According to some embodiments, a method of repairing a fractured bone (e.g., the fractured bone 100) may include positioning the intramedullary nail 117 in an intramedullary canal 110 (e.g., the hole 108) of the fractured bone 100 such that a portion of the intramedullary nail 117 is in the first bone fragment 102 of the fractured bone 100, another portion of the intramedullary nail 117 is in the second bone fragment 104 of the fractured bone 100, and the intramedullary nail 117 extends across the fracture 106 between the first bone fragment 102 and the second bone fragment 104.

For example, when inserting the multi-lumen cannula 120 through the recess 180 of the targeting guide 182, the targeting guide 182 may be secured to the intramedullary nail 117, which may be positioned within the hole 108 formed in the fractured bone 100. The multi-lumen cannula 120 may be advanced toward the intramedullary nail 117, according to the method, until the multi-lumen cannula 120 contacts the skin of the patient or the fractured bone 100.

When the intramedullary nail 117 is positioned within the hole 108 formed in the fractured bone 100, the method may include advancing the first bone fixator 190 into the first bone fragment 102 (e.g., a distal fragment of a fibula) and advancing the second bone fixator 192 into the second bone fragment 104. As shown in FIG. 17, a force F may be applied to the multi-lumen cannula 120, the targeting guide 182, the first bone fixator 190, the second bone fixator 192, or any combination thereof. When the first bone fixator 190 is inserted into and secured to the first bone fragment 102 (e.g., a distal fragment of a fibula) and the second bone fixator 192 is inserted into and secured to the second bone fragment 104, the force F moves the first bone fragment 102 away from the second bone fragment 104.

Thus, the method may include increasing a length of the fractured bone 100 with the multi-lumen cannula 120 used as a fracture reduction sleeve. The method may include rotating the first bone fragment 102 relative to the second bone fragment 104 of the fractured bone 100 (e.g., to realign the first bone fragment 102 and the second bone fragment 104 to match the pre-fractured condition of the fractured bone 100). However, the multi-lumen cannula 120 may be used in other procedures involving the insertion of bone fixators along desired pathways. Implantation of the intramedullary nail 117 and methods of bone fixation described herein may be performed without the use of the multi-lumen cannula 120. For example, temporary fixators (e.g., K-wires) may be inserted into the first bone fragment 102 and/or the second bone fragment 104 (either with or without the use of the targeting guide 182 and with or without the use of the multi-lumen cannula 120). When inserted, force may be applied to the temporary fixators to manipulate the first bone fragment 102 relative to the second bone fragment 104.

When the intramedullary nail 117 is positioned within the hole 108 formed in the fractured bone 100, the method may include securing the intramedullary nail 117 to the first bone fragment 102 via insertion of the third bone fixator 194. Transitioning the deployable bone fixator 196 may include abutting the movable talons 326 with the inner surface of the second bone fragment 104 of the fractured bone 100. Advancing the actuator 186 along the length of the intramedullary nail 117 may include moving a tip 187 of the actuator 186 across the fracture 106 and passing the tip 187 between the first bone fixator 190 and the second bone fixator 192.

As shown in FIG. 17, when the fractured bone 100 has the desired length (e.g., while the force F is being applied to the first bone fragment 102 (e.g., via the multi-lumen cannula 120, the targeting guide 182, the first bone fixator 190, the second bone fixator 192, or any combination thereof) the first bone fixator 190 and the second bone fixator 192 may be advanced further (e.g., medially into the body) to enter a second bone (e.g., a tibia 109 and/or a talus 111). Thus the method may include securing a position of the first bone fragment 102 to another bone (e.g., not the fractured bone 100 or any of its fragments). When the first bone fragment 102 is secured to the other bone, the force F may be removed as it is no longer needed to maintain the desired length of the fractured bone 100, and the position of the first bone fragment 102 relative to the second bone fragment 104.

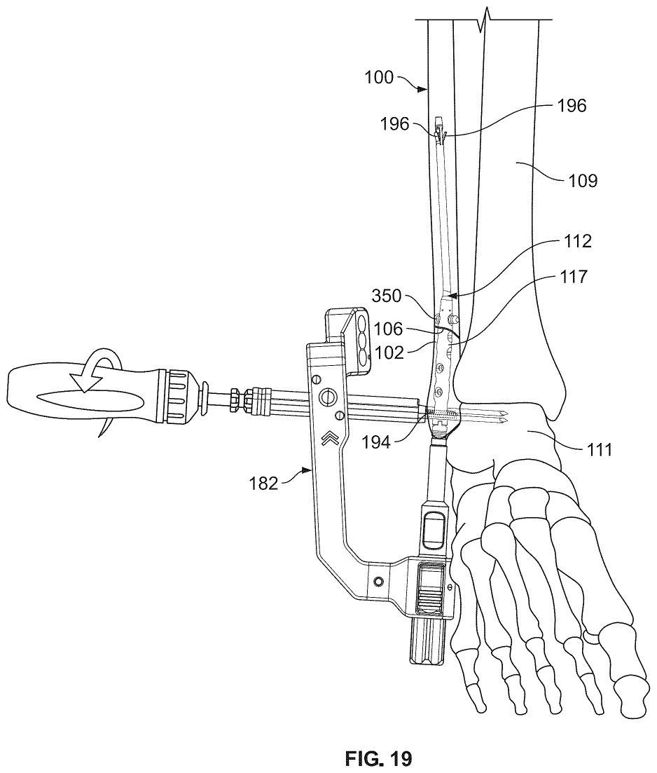

As shown in FIG. 18, rotating the actuator 186 within the intramedullary nail 117 may deploy the one or more talons 188 thereby securing the intramedullary nail 117 to the second bone fragment 104. After deploying the one or more talons 188, the actuator 186 may be withdrawn entirely from the intramedullary canal 110 of the intramedullary nail 117 prior to advancing the third bone fixator 194 into the bone fastener receiving hole 184, as shown in FIG. 19. Inserting the third bone fixator 194, according to the method, may secure the intramedullary nail 117 to the first bone fragment 102. The bone screws inserted through the transverse fixation holes 314, 334 of the intramedullary nail 117 may be inserted in different orders. For example, the bone screw 350 may be inserted through the transverse fixation hole 334 of the intermediate body portion 330 prior to insertion of any bone screws being inserted through the transverse fixation holes 314 of the proximal body portion 310. Alternatively, the bone screw 350 may be inserted through the transverse fixation hole 334 of the intermediate body portion 330 after insertion of any/all bone screws being inserted through the transverse fixation holes 314 of the proximal body portion 310.

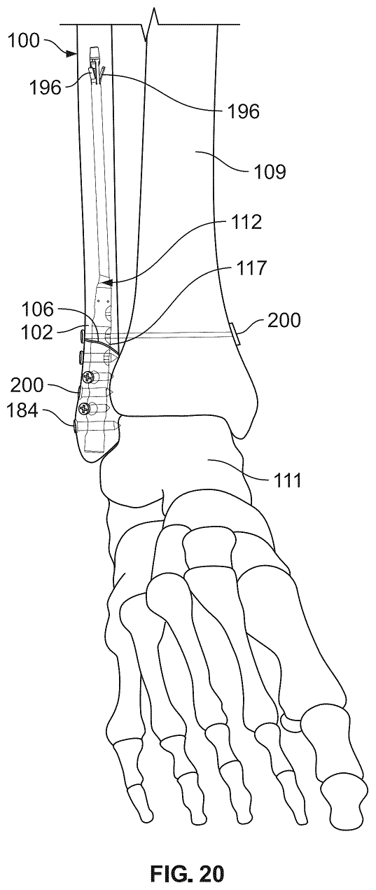

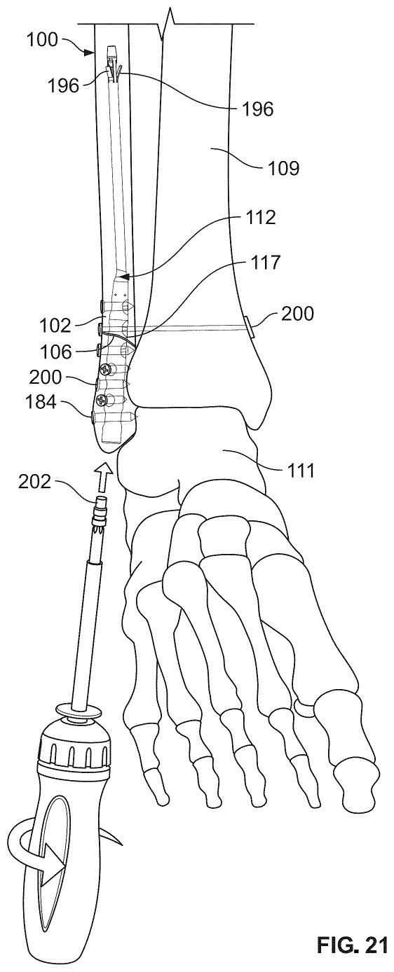

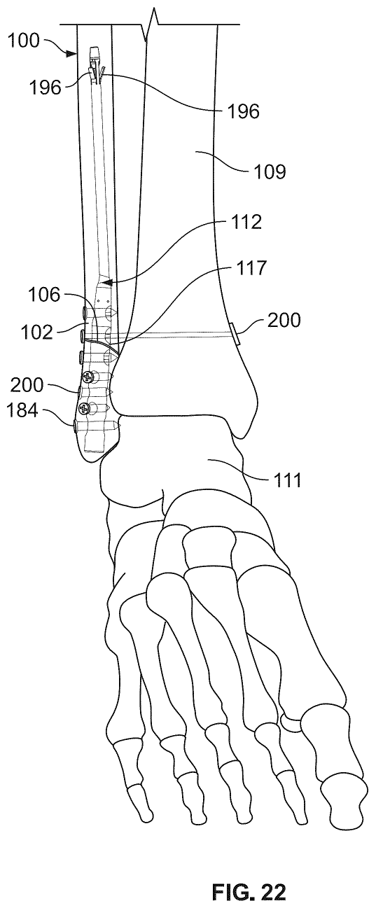

As shown in FIG. 20, additional permanent bone fixators 200 (e.g., bone screws similar to the bone screw 350) may be inserted into respective ones of the plurality of transverse fixation holes 314 to secure the proximal body portion 310 of the intramedullary implant 112 to the first bone fragment 102 of the fractured bone 100. According to some embodiments, all of the additional permanent bone fixators 200 may be inserted into corresponding bone fastener receiving holes to secure the intramedullary implant 112 to the first bone fragment 102 of the fractured bone 100. As shown in FIG. 21, after insertion of the additional permanent bone fixators 200 (e.g., when the intramedullary nail 117 is fully secured to the fractured bone 100), an end cap 202 may be coupled to the intramedullary nail 117 to close the path through which the actuator 186 passed. As shown in FIG. 22, the completed intramedullary nail 117 and the permanent bone fixators remain secured to the fractured bone 100, and the temporary bone fixators may be removed.

Although described above in use to fixate first and second fragments of a fractured bone, the intramedullary implant 112 and its methods of use are not so limited. The fractured bone 100 may comprise more than two (e.g., three, four, five, etc.) bone fragments that need to be repositioned and secured to restore the fractured bone 100 to its pre-fracture condition (e.g., a butterfly fracture).

According to some embodiments, the deployable fixation element 324 may be secured within one bone fragment, one or more of the plurality of transverse fixation holes 314 may be secured within another bone fragment, and the transverse fixation hole 334 of the intermediate body portion 330 may be secured within a third bone fragment. For example, a method of fixating multiple fragments of a fractured bone relative to one another may comprise advancing the distal body portion 320 through an opening formed in a first bone fragment of the fractured bone 100. The method may further comprise advancing the distal body portion 320 through a medullary cavity of the first bone fragment and advancing the distal body portion 320 into and through a portion of a medullary cavity of a second bone fragment of the fractured bone 100.

The method may further comprise advancing the proximal body portion 310 of the intramedullary implant 112 through the opening formed in the first bone fragment and advancing the proximal body portion 310 into the medullary cavity of the first bone fragment. The method may comprise deploying the deployable fixation element 324 of the distal body portion 320 within the medullary cavity of the second bone fragment until the fixation element contacts an inner wall of the second bone fragment that defines the medullary cavity of the second bone fragment. A first bone fixation element may be inserted through an exterior surface of a third bone fragment of the fractured bone 100 that is between the first and second bone fragments of the fractured bone 100 and through the transverse fixation hole 334 defined by the intermediate body portion 330 of the intramedullary implant 112. A second bone fixation element may be inserted through an exterior surface of the first bone fragment and through one of the plurality of transverse fixation holes 314 defined by the proximal body portion 310 of the intramedullary implant 112.

The above description of illustrated embodiments, including what is described in the Abstract, is not intended to be exhaustive or to limit the embodiments to the precise forms disclosed. Although specific embodiments of and examples are described herein for illustrative purposes, various equivalent modifications can be made without departing from the spirit and scope of the disclosure, as will be recognized by those skilled in the relevant art. The various embodiments described above can be combined to provide further embodiments.

Many of the methods described herein can be performed with variations. For example, many of the methods may include additional acts, omit some acts, and/or perform acts in a different order than as illustrated or described.

As used herein, “greater than” and “less than” limits may also include the number associated therewith. Stated another way, “greater than” and “less than” may be interpreted as “greater than or equal to” and “less than or equal to.” It is contemplated that this language may be subsequently modified in the claims to include “or equal to.” For example, “greater than 2 mm” may be interpreted as, and subsequently modified in the claims as “greater than or equal to 2 mm.”

In some embodiments, any or some of the components or steps disclosed herein may be considered optional. In some cases, the disclosed embodiments may expressly exclude any or some of the aforementioned elements or steps in this description, e.g., via claim language. For example, claim language may be modified to recite that the disclosed intramedullary implant and/or methods, etc., do not utilize or comprise a deployable fixation element or a multi-lumen cannula. Such negative limitations are contemplated, and this text serves as support for negative limitations for components, steps, and/or features.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

1. An intramedullary implant comprising:
a proximal body portion that extends along a first central axis, the proximal body portion defining a plurality of transverse fixation holes that each extend through the proximal body portion and intersect the first central axis, wherein the proximal body portion defines a first maximum cross-sectional dimension measured in a first plane normal to the first central axis;
a distal body portion that extends along a second central axis that is angularly offset from the first central axis, the distal body portion comprising a deployable fixation element that transitions from an insertion configuration to a deployed configuration in which a portion of the deployable fixation element is farther from the second central axis than the portion is from the second central axis in the insertion configuration, wherein the distal body portion defines a second maximum cross-sectional dimension measured in a second plane normal to the second central axis, and the second maximum cross-sectional dimension is less than the first maximum cross-sectional dimension; and
an intermediate body portion between the proximal body portion and the distal body portion, the intermediate body portion defining a transverse fixation hole that extends through the proximal body portion and intersects the first central axis, wherein the intermediate body portion defines a third maximum cross-sectional dimension measured in a third plane normal to the first central axis, and the third maximum cross-sectional dimension tapers as the intermediate body portion approaches the distal body portion.

2. The intramedullary implant of claim 1 wherein the proximal body portion defines:
a first transverse fixation hole that intersects the first central axis at a location proximal of the transverse fixation hole defined by the intermediate body portion such that the first transverse fixation hole is angularly offset from the transverse fixation hole defined by the intermediate body portion; and
a second transverse fixation hole that intersects the first central axis at a location proximal of the transverse fixation hole defined by the intermediate body portion and distal of the first transverse fixation hole such that the second transverse fixation hole is angularly offset from both the first transverse fixation hole and the transverse fixation hole defined by the intermediate body portion.

3. The intramedullary implant of claim 2, further comprising a third transverse fixation hole that extends through the proximal body portion parallel to the first transverse fixation hole and that intersects the first central axis at a location proximal of the transverse fixation hole defined by the intermediate body.

4. The intramedullary implant of claim 3 wherein the second transverse fixation hole intersects the first central axis at a location proximal of the third transverse fixation hole and distal of the first transverse fixation hole.

5. The intramedullary implant of claim 4, further comprising a fourth transverse fixation hole that extends through the proximal body portion parallel to the second transverse fixation hole and that intersects the first central axis at a location proximal of the transverse fixation hole defined by the intermediate body.

6. The intramedullary implant of claim 5 wherein the fourth transverse fixation hole intersects the first central axis at a location proximal of the transverse fixation hole defined by the intermediate body and distal of the third transverse fixation hole.

7. The intramedullary implant of claim 1, further comprising:
an axial bore that extends through the proximal body portion along the first central axis, through the intermediate body portion, and into the distal body portion,
wherein the axial bore intersects the transverse fixation hole defined by the intermediate body portion and any transverse fixation holes defined by the proximal body portion.

8. The intramedullary implant of claim 7, further comprising:
an actuator sized to advance along the axial bore and engage the deployable fixation element and transitions the deployable fixation element from the insertion configuration to the deployed configuration.

9. The intramedullary implant of claim 1, further comprising:
a set of three transverse fixation holes that are parallel to one another, and that each intersect the first central axis,
wherein the transverse fixation hole defined by the intermediate body portion is one of the set of three transverse fixation holes.

10. The intramedullary implant of claim 1 wherein the distal body portion is devoid of any transverse fixation holes.

11. An intramedullary implant comprising:
a proximal body portion that extends along a first central axis, the proximal body portion defining a first transverse fixation hole that extends through the proximal body portion and intersects the first central axis, a second transverse fixation hole that extends through the proximal body portion and intersects the first central axis and is angularly offset from the first transverse fixation hole, and a set of three transverse fixation holes that each extend through the proximal body portion parallel to one another and intersect the first central axis, wherein the set of three transverse fixation holes are each angularly offset from both the first transverse fixation hole and the second transverse fixation hole; and
a distal body portion that extends along a second central axis that is angularly offset from the first central axis, the distal body portion comprising a deployable fixation element that transitions from an insertion configuration to a deployed configuration in which a portion of the deployable fixation element is farther from the second central axis than the portion is from the second central axis in the insertion configuration,
wherein each of the set of three transverse fixation holes are positioned distally of both the first transverse fixation hole and the second transverse fixation hole.

12. The intramedullary implant of claim 11, further comprising a third transverse fixation hole that extends through the proximal body portion parallel to the first transverse fixation hole and intersects the first central axis at a location proximal of the set of three transverse fixation holes.

13. The intramedullary implant of claim 12 wherein the second transverse fixation hole intersects the first central axis at a location proximal of the third transverse fixation hole and distal of the first transverse fixation hole.

14. The intramedullary implant of claim 13, further comprising a fourth transverse fixation hole that extends through the proximal body portion parallel to the second transverse fixation hole and intersects the first central axis at a location proximal of the set of three transverse fixation holes.

15. The intramedullary implant of claim 14 wherein the fourth transverse fixation hole intersects the first central axis at a location proximal of the set of three transverse fixation holes and distal of the third transverse fixation hole.

16. The intramedullary implant of claim 11 wherein the proximal body portion defines a first maximum cross-sectional dimension measured in a first plane normal to the first central axis, the distal body portion defines a second maximum cross-sectional dimension measured in a second plane normal to the second central axis, and the second maximum cross-sectional dimension is less than the first maximum cross-sectional dimension.

17. The intramedullary implant of claim 11, further comprising:
an intermediate body portion between the proximal body portion and the distal body portion, the intermediate body portion defining a transverse fixation hole that extends through the proximal body portion and intersects the first central axis,
wherein the intermediate body portion defines a third maximum cross-sectional dimension measured in a third plane normal to the first central axis, and the third maximum cross-sectional dimension tapers as the intermediate body portion approaches the distal body portion.

18. The intramedullary implant of claim 11, further comprising:
an axial bore that extends through the proximal body portion along the first central axis and into the distal body portion,
wherein the axial bore intersects the first transverse fixation hole, the second transverse fixation hole, and each of the set of three transverse fixation holes.

19. The intramedullary implant of claim 18, further comprising:
an actuator sized to advance along the axial bore and engage the deployable fixation element and transitions the deployable fixation element from the insertion configuration to the deployed configuration.

20. A method of fixating a first bone fragment relative to a second bone fragment, the method comprising:
advancing a distal body portion of an intramedullary implant through an opening formed in the first bone fragment;
advancing the distal body portion through a medullary cavity of the first bone fragment;
moving the distal body portion across a fracture site between the first bone fragment and the second bone fragment;
advancing the distal body portion into and through a portion of a medullary cavity of the second bone fragment;
advancing a proximal body portion of the intramedullary implant through the opening formed in the first bone fragment;
advancing the proximal body portion into the medullary cavity of the first bone fragment;
deploying a fixation element of the distal body portion within the medullary cavity of the second bone fragment until the fixation element contacts an inner wall of the second bone fragment that defines the medullary cavity of the second bone fragment;
inserting a first bone fixation element through an exterior surface of the second bone fragment and through a first transverse fixation hole defined by the intramedullary implant; and
inserting a second bone fixation element through an exterior surface of the first bone fragment and through a second transverse fixation hole defined by the intramedullary implant.

21. The method of claim 20, further comprising:
manipulating the first bone fragment relative to the second bone fragment when the distal body portion is within the medullary cavity of the second bone fragment and the proximal body portion is within the medullary cavity of the first bone fragment.

22. The method of claim 20 wherein:
inserting the first bone fixation element comprises rotating the first bone fixation element about a first fixation element axis; and
inserting the second bone fixation element comprises rotating the second bone fixation element about a second fixation element axis that is angularly offset from the first fixation element axis.

23. The method of claim 22, further comprising:
inserting a third bone fixation element through an exterior surface of the first bone fragment and through a third transverse fixation hole defined by the intramedullary implant,
wherein the third bone fixation element includes a bone screw or a flexible fixation device.

24. The method of claim 23 wherein inserting the third bone fixation element includes rotating the third bone fixation element about a third fixation element axis that is parallel to the first fixation element axis.

25. The method of claim 23 wherein inserting the third bone fixation element includes rotating the third bone fixation element about a third fixation element axis that is angularly offset from both the first fixation element axis and the second fixation element axis.

26. The method of claim 20, further comprising:
advancing an actuator through an axial bore of the intramedullary implant when the distal body portion is within the medullary cavity of the second bone fragment and the proximal body portion is within the medullary cavity of the first bone fragment,
wherein the axial bore intersects the first transverse fixation hole and the second transverse fixation hole, and
wherein deploying the fixation element of the distal body portion comprises engaging the fixation element with a portion of the actuator positioned within the axial bore at a location within the medullary cavity of the second bone fragment.

27. The method of claim 20 wherein inserting the first bone fixation element through the exterior surface of the second bone fragment and through the first transverse fixation hole defined by the intramedullary implant comprises:
inserting the first bone fixation element through an intermediate body portion of the intramedullary implant between the proximal body portion and the distal body portion,
wherein the intermediate body portion tapers toward the distal body portion.

28. A method of fixating multiple fragments of a fractured bone relative to one another, the method comprising:
advancing a distal body portion of an intramedullary implant through an opening formed in a first bone fragment of the fractured bone;
advancing the distal body portion through a medullary cavity of the first bone fragment;
advancing the distal body portion into and through a portion of a medullary cavity of a second bone fragment of the fractured bone;
advancing a proximal body portion of the intramedullary implant through the opening formed in the first bone fragment;
advancing the proximal body portion into the medullary cavity of the first bone fragment;
deploying a fixation element of the distal body portion within the medullary cavity of the second bone fragment until the fixation element contacts an inner wall of the second bone fragment that defines the medullary cavity of the second bone fragment;
inserting a first bone fixation element through an exterior surface of a third bone fragment of the fractured bone and through a first transverse fixation hole defined by the intramedullary implant, wherein the third bone fragment is between the first bone fragment and the second bone fragment; and
inserting a second bone fixation element through an exterior surface of the first bone fragment and through a second transverse fixation hole defined by the intramedullary implant.

29. The method of claim 28, further comprising:
moving the distal body portion across a fracture site between the first bone fragment and the third bone fragment; and
moving the distal body portion across a fracture site between the third bone fragment and the second bone fragment.

30. The method of claim 29 wherein inserting the first bone fixation element through the exterior surface of the third bone fragment and through the first transverse fixation hole defined by the intramedullary implant comprises:
inserting the first bone fixation element through an intermediate body portion of the intramedullary implant between the proximal body portion and the distal body portion,
wherein the intermediate body portion tapers toward the distal body portion.
